(19) United States
(12) Patent Application Publication
(10) Pub. No.: US 2024/0294479 A1
(43) Pub. Date: Sep. 5, 2024

(54) POLYMORPHS OF 2-(3,5-DICHLOROPHENYL)-L,3-BENZOXAZOLE-6-CARBOXYLIC ACID

(71) Applicant: HONOUR LAB LIMITED, Hyderabad (IN)

(72) Inventors: Kura RATHNAKAR REDDY, Hyderabad (IN); Kesireddy SUBHASH CHANDER REDDY, Hyderabad (IN); Mule SIVA NAGI REDDY, Hyderabad (IN)

(21) Appl. No.: 18/570,860

(22) PCT Filed: Jun. 16, 2022
(86) PCT No.: PCT/IB2022/055565

§ 371 Date: Dec. 15, 2023

(30) Foreign Application Priority Data
Jun. 16, 2021 (IN) 202141026745

(51) Int. Cl. C07D 263/57; A61K 31/423

(52) U.S. Cl. CPC C07D 263/57; A61K 31/423

(57) ABSTRACT

The present invention relates to polymorphic forms of Tafamidis of Formula I and process for their preparation and pharmaceutical composition comprising it.

### FIELD OF THE INVENTION

The present invention relates to polymorphs of 2-(3,5-Dichlorophenyl)-1,3-benzoxazole-6-carboxylic acid, process for their preparation and pharmaceutical composition comprising it.

### BACKGROUND OF THE INVENTION

Tafamidis and Tafamidis Meglumine represented by the following structures Formula I and Formula IA that contains Tafamidis as active moiety chemically known as 2-(3,5-dichlorophenyl)-1,3-benzoxazole-6-carboxylic acid. In USA, Tafamidis is approved as both Tafamidis and Tafamidis Meglumine salt, whereas in Europe it's approved as Tafamidis Meglumine salt and are marketed under trade name VYNDAMAX™ and VYNDAQEL®. Tafamidis and its salts are approved and indicated for Transthyretin stabilizers for the treatment of the cardiomyopathy of wild type or hereditary transthyretin-mediated amyloidosis in adults to reduce cardiovascular mortality and cardiovascular-related hospitalization. The recommended dosage is either VYNDAQEL 80 mg (four 20-mg tafamidis meglumine capsules) orally once daily, or VYNDAMAX 61 mg (one 61-mg tafamidis capsule) orally once daily.

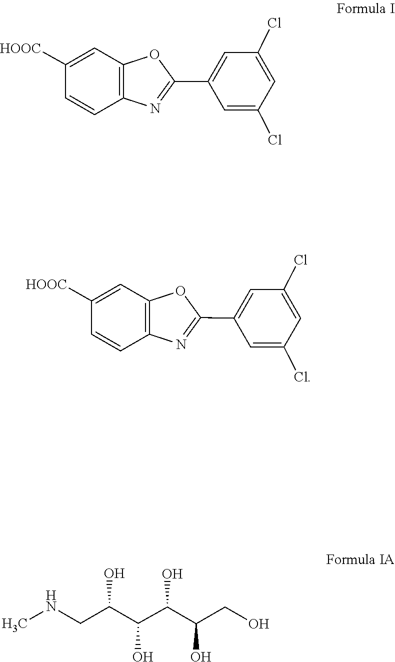 [SMILES: O=C(O)c1ccc2nc(-c3cc(Cl)cc(Cl)c3)oc2c1 ; O=C(O)c1ccc2nc(-c3cc(Cl)cc(Cl)c3)oc2c1 ; CNC[C@H](O)[C@@H](O)[C@H](O)[C@H](O)CO]

U.S. Pat. No. 7,214,695 discloses 6-Carboxy-2-(3,5-dihalophenyl)-benzoxazole compounds or pharmaceutically acceptable salts thereof. U.S. Pat. No. '695 disclose the process for the preparation of Tafamidis as follows:

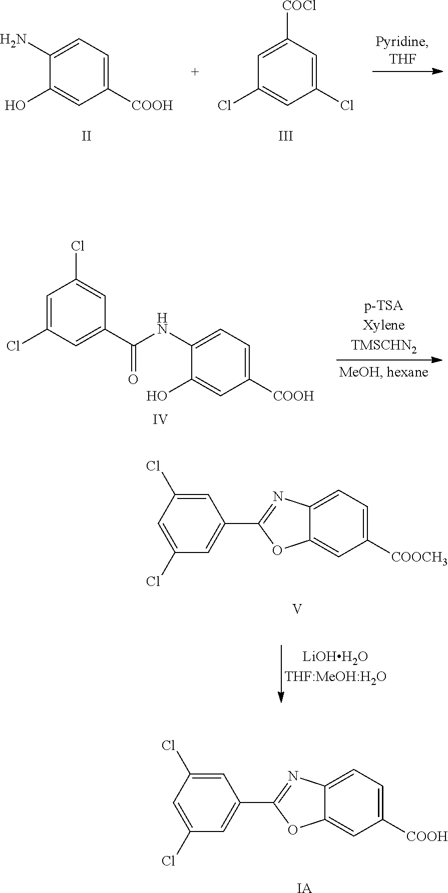

U.S. Pat. No. 9,249,112 B2 of Pfizer Inc, discloses Tafamidis meglumine different polymorphic forms, Form M, Form B and amorphous Form A.

U.S. Pat. No. 9,770,441 B1 of Pfizer Inc discloses different polymorphic forms of Tafamidis designated as Form 1, Form 2, Form 4, Form 6, amorphous form; wherein Tafamidis Form 1 has been prepared from Isopropanol; Form 2 has been prepared by evaporating tetrahydrofuran solution of Form 1; Form 4 has been prepared by heating suspension of Form 1 in tetrahydrofuran and filtering the hot solution in to toluene and then storing in freezer overnight; Form 6 has been prepared by heating suspension of Form 1 in tetrahydrofuran, adding dimethylacetamide and the solution resulted was transferred in to dichloromethane in chilled ice/water bath and then by vacuum filtration and drying.

WO 2019175263 A1 of Azad Pharma Ag discloses Tafamidis crystalline form, Tafamidis acetic acid adduct, Tafamidis formic acid adduct; wherein Tafamidis crystalline form is prepared by heating dispersion of Tafamidis acetic acid adduct in solvent capable of removing acetic acid and thereafter drying the precipitate obtained.

WO 2020232325 A1 of Teva discloses different polymorphic forms designated as Tafamidis Form I, II, III, IV, V, amorphous form; wherein Form I, a hydrate form has been prepared from amorphous form and also from tetrahydrofuran and water; Form II, a 2-methyl tetrahydrofuran solvate has been prepared by evaporating a solution of Tafamidis in 2-methyl tetrahydrofuran; Form III, Form IV are acetic acid solvates, prepared from acetic acid; Form V is a methanol solvate obtained from solvent antisolvent crystallization, wherein antisolvent taken is methanol and methanol solvate is dried to give Form V in anhydrous form.

WO 2021001858 A1 discloses different polymorphic forms designated as Tafamidis Form S, Form N, Form R; wherein Form S, an acetic acid solvate has been prepared from acetic acid; Form N has been prepared by treating Tafamidis Form S with water and then isolated; Form R has been prepared by using polar aprotic solvent and water as anti-solvent and further treated with acetic acid and isolated.

Polymorphism is defined as “the ability of a substance to exist as two or more crystalline phases that have different arrangement and/or conformations of the molecules in the crystal Lattice. Thus, in the strict sense, polymorphs are different crystalline structures of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning calorimetry (DSC) and Infrared spectrometry (IR). Tafamidis exits in different polymorphic forms which differ in the physicochemical properties like dissolution and solubility, chemical and physical stability, flowability and hygroscopicity as well as preparation thereof. However, there is still a need of other polymorphs and salts, which are stable and suitable for pharmaceutical composition with higher solubility.

The present inventors have developed the ecofriendly process for preparation of Tafamidis crystalline forms from Tafamidis meglumine by avoiding use of environmentally hazardous solvents.

In view of this the present inventors have found crystalline forms of Tafamidis which are stable, reproducible, free of other polymorphic forms and suitable for pharmaceutical composition as well as a process suitable industrially for preparing Tafamidis and its pharmaceutically acceptable salt thereof.

### OBJECTIVES OF THE INVENTION

The objective of the present invention is to provide crystalline forms of Tafamidis, processes for their preparation and pharmaceutical composition comprising it.

Another objective of the present invention is to provide crystalline forms of Tafamidis with high yields and high purity.

Another objective of the present invention is to provide process for crystalline forms of Tafamidis from Tafamidis meglumine without using organic solvents.

### SUMMARY OF THE INVENTION

The present invention relates to Tafamidis crystalline form designated as Form HN1 having Powder X-Ray Diffraction pattern as shown in FIG. 1.

In other embodiment, the present invention relates to a process for the preparation of Tafamidis crystalline Form HN1 having Powder X-Ray Diffraction pattern as shown in FIG. 1, which comprises:
a. treating Tafamidis Meglumine with water;
b. adjusting the pH of the mass with an acid to 3-4.5 to precipitate the solid; and
c. filtering the solid and drying the solid at 80-85° C. till the moisture content is <1.0% w/w,
   wherein acid is selected from group comprising of hydrochloric acid, sulfuric acid, acetic acid, formic acid, trifluoroacetic acid, methanesulfonic acid, citric acid.

In other embodiment, the present invention relates to Tafamidis crystalline form designated as Form HN2 having Powder X-Ray Diffraction pattern as shown in FIG. 2.

In other embodiment, the present invention relates to a process for the preparation of Tafamidis crystalline Form HN2 having Powder X-Ray Diffraction pattern as shown in FIG. 2, which comprises:
a. treating Tafamidis Meglumine with water;
b. adjusting the pH of the mass with an acid to 3-4.5 to precipitate the solid; and
c. filtering the solid and drying the solid below 50° C. till the moisture content is 4-6% w/w,
   wherein acid is selected from group comprising of hydrochloric acid, sulfuric acid, acetic acid, formic acid, trifluoroacetic acid, methanesulfonic acid, citric acid.

In other embodiment, the present invention relates to Tafamidis crystalline form designated as Form HN3 having Powder X-Ray Diffraction pattern as shown in FIG. 3.

In other embodiment, the present invention relates to a process for the preparation of Tafamidis crystalline Form HN3 having Powder X-Ray Diffraction pattern as shown in FIG. 3, which comprises:
a. treating Tafamidis Meglumine with water;
b. adjusting the pH of the mass with an acid to 3-5 to precipitate the solid; and
c. filtering the solid and drying the solid at below 40° C.,
   wherein acid is selected from group comprising of hydrochloric acid, sulfuric acid, acetic acid, formic acid, trifluoroacetic acid, methanesulfonic acid, citric acid.

### BRIEF DESCRIPTION OF THE DRAWINGS

FIG. 1: X-ray powder diffraction spectrum of Tafamidis crystalline Form HN1.

FIG. 2: X-ray powder diffraction spectrum of Tafamidis crystalline Form HN2.

FIG. 3: X-ray powder diffraction spectrum of Tafamidis crystalline Form HN3.

X-ray powder diffraction spectrum was measured on a Bruker axs D8 advance X-ray powder diffractometer having a copper-a radiation. Adequate sample was gently flattered on a sample holder and scanned from 2 to 50 degrees two-theta, at 0.02 increment and scan speed of 0.2 Sec/Step. The sample was simply placed on the sample holder. The sample was rotated at 30 rpm at a voltage 40 KV and current 35 mA.

### DETAILED DESCRIPTION OF THE INVENTION

The present invention relates to different crystalline forms of Tafamidis designated as Form HN1, Form HN2 and Form HN3.

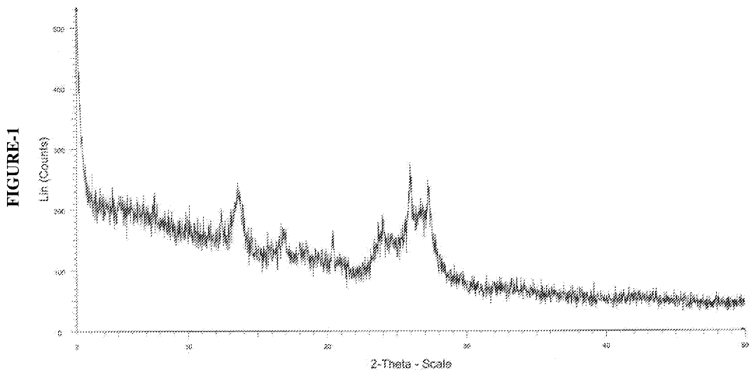

In one aspect, the present invention relates to Tafamidis crystalline form designated as Form HN1 having Powder X-Ray Diffraction pattern as shown in FIG. 1.

In another aspect, the present invention relates to Tafamidis crystalline Form HN1 having Powder X-Ray Diffraction pattern as shown in FIG. 1, which comprises treating tafamidis Meglumine with water and adjusting the pH of the mass with an acid to 3-4.5 wherein acid is selected from group comprising of hydrochloric acid, sulfuric acid, acetic acid, formic acid, trifluoroacetic acid, methanesulfonic acid, citric acid to precipitate the solid, filtering the solid and drying the solid at 80-85° C. for 12-14 hours till the moisture content is <1.0% w/w.

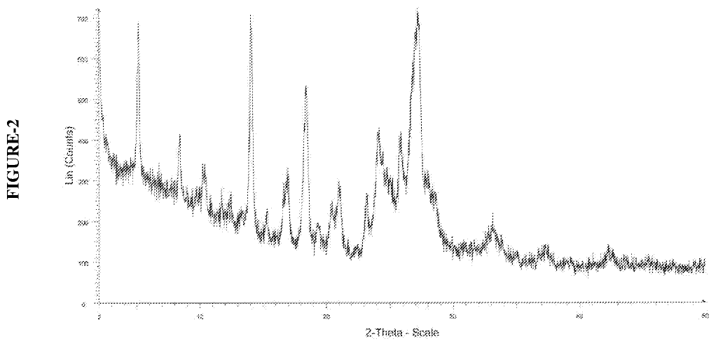

In another aspect, the present invention relates to Tafamidis crystalline form designated as Form HN2 having Powder X-Ray Diffraction pattern as shown in FIG. 2.

In another aspect, the present invention relates to Tafamidis crystalline Form HN2 having Powder X-Ray Diffraction pattern as shown in FIG. 2, which comprises treating tafamidis Meglumine with water and adjusting the pH of the mass with an acid to 3-4.5 wherein acid is selected from group comprising of hydrochloric acid, sulfuric acid, acetic acid, formic acid, trifluoroacetic acid, methanesulfonic acid, citric acid to precipitate the solid, filtering the solid and drying the solid below 50° C. for 18-24 hours till the moisture content is 4-6% w/w.

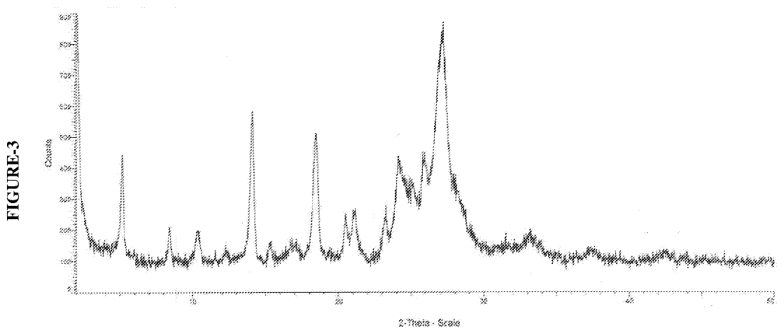

In another aspect, the present invention relates to Tafamidis crystalline form designated as Form HN3 having Powder X-Ray Diffraction pattern as shown in FIG. 3.

In another aspect, the present invention relates to Tafamidis crystalline Form HN3 having Powder X-Ray Diffraction pattern as shown in FIG. 3, which comprises treating tafamidis Meglumine with water and adjusting the pH of the mass with an acid to 3-5 wherein acid is selected from group comprising of hydrochloric acid, sulfuric acid, acetic acid, formic acid, trifluoroacetic acid, methanesulfonic acid, citric acid to precipitate the solid, filtering the solid and drying the solid below 40° C. for 14-15 hours till the moisture content is less than 10%.

In another aspect, the present invention provides drying the solid is carried out by using hot air or under vacuum.

In another aspect, the present invention provides a pharmaceutical composition comprising an oral dosage form of Tafamidis crystalline Form HN1, Form HN2 or Form HN3 wherein oral dosage form is a tablet, pill or capsule preferably soft gel capsule.

In another aspect, the crystalline forms of the present invention are preserved in air tight containers and stored room temperature and preferably at 2-8° C. temperature under nitrogen atmosphere.

In another aspect, the room temperature is considered at 25-30° C.

In another aspect, the crystalline forms of the present invention are packed in laminated polybags filled with nitrogen.

In another aspect, the crystalline forms of the present invention are protected from heat, light and moisture.

In another aspect, the present invention, Tafamidis crystalline polymorphs of the present invention can be prepared from Tafamidis salts selected from Tafamidis sodium, Tafamidis potassium, Tafamidis triethanolamine.

EMEA scientific discussion provides the solubility studies with Tafamidis (Form 1) showed that the active substance has water solubility <2 μg/mL at pH 5 and below. However the crystalline Forms of the present invention has water solubility greater than or equal to 3 μg/mL at pH 5 and below.

The solubility of Tafamidis crystallin Form HN3 in water at different pH is as follows:

| pH | Form HN3 |
|---|---|
| 3 | 3 μg/mL |
| 4 | 3 μg/mL |
| 5 | 3 μg/mL |

The yield reported for Form 1 in U.S. Pat. No. 9,770,441 is 82-89% wt/wt and purity is about 95% however crystalline Forms of the present invention have yields greater than 98% wt/wt and purity greater than 99% by HPLC.

The present invention is advantageous over prior art by
Avoiding use of solvents in the preparation of crystalline polymorphs of Tafamidis
Higher solubility when compared to innovator Tafamidis Form 1
Higher yields and purities

In another aspect of the present invention Tafamidis meglumine has been prepared by following the process known in the prior art.

In the following section embodiments are described by way of examples to illustrate the process of invention. However, these do not limit the scope of the present invention. Variants of these examples would be evident to persons ordinarily skilled in the art.

### EXAMPLES

### Reference Example

### Preparation of Tafamidis

4-amino-3-hydroxybenzoicacid (1.0 eq, LR) was dissolved at 20° C. in a mixture of tetrahydrofuran (19 L/kg) and water (1.9 L/Kg). 3,5-dichlorobenzoylchloride (1.3 equiv) was added as a tetrahydrofuran solution (1.9 L/kg) and the mixture stirred for at least 30 minutes at 20° C. Once the reaction was deemed complete by HPLC (<5% remaining 4-amino-3-hydroxybenzoicacid), triethylamine (1.2 equiv) was added and the mixture was heated to 35° C. and stirred for at least 90 minutes. The solvent was partially displaced with ethanol by constant level distillation until 5-15% THF remained. The slurry was cooled to 20° C. and stirred for at least 60 minutes then the slurry was filtered. The solids were washed with ethanol (3×4 L/kg) then dried under vacuum at 65° C. for at least 16 hours to give pure 4-[(3,5-dichlorobenzoyl)amino]-3-hydroxybenzoic acid in 88-92% yield.

To a slurry of 4-[(3,5-dichlorobenzoyl)amino]-3-hydroxybenzoic acid (1.0 equiv) in tetrahydrofuran (10 L/kg) was added triethylamine (1.1 equiv), followed by water (4 equiv). The mixture was held at 20-25° C. for 1 hour, then the mixture was filtered to remove any remaining insoluble material. Methanesulfonic acid (1.6 equiv) was added and a slurry formed. A constant level displacement of THF/water with toluene was carried out until the reaction temperature was at least 107° C., at which point the displacement was stopped and the reaction then refluxed for at least 15 hours. Once the reaction was deemed complete by UPLC, i.e. >95% pure, it was cooled to 20° C. and 2-propanol (5 L/kg) was added. The slurry was granulated for at least 60 minutes, then filtered and washed twice with 2-propanol (4 I/kg each wash) and dried under vacuum at 60-70° C. for a minimum of 18 hours to give Form 1 in 82-89% yield.

### Preparation of Tafamidis Meglumine

6-Carboxy-2-(3,5-dichlorophenyl)-benzoxazole free acid (2.5 g, 8.1 mmol) and 2-propanol (49 mL) were charged to a 100 mL jacketed, 2-neck round bottom flask with magnetic stirrer. The resulting slurry was warmed to 70° C. with stirring. Water (8.8 mL) was then charged. In a separate 15 mL round bottom flask a solution of N-methyl-D-glucamine (1.58 g, 8.1 mmol) in 5 mL water was prepared and dissolved with stirring. The aqueous N-methyl-D-glucamine solution was then transferred to the reaction flask over 2 min. Most (but not all) of the solids dissolved by the end of this addition. After 5 min stirring and warming to 79° C., a clear, pale yellow solution resulted. The solution was filtered through a bed of Celite™, cooled to 60° C., then cooled to 10° C. over 2 h. The resulting solids were collected by filtration, washing with 10 mL of 2-propanol. 3.35 g product was obtained (82% yield).

### Example 1

### Preparation of Tafamidis Form HN1

Charged Tafamidis Meglumine (100 gm) and DM water (1500 ml) and cooled the mass to 10-20° C. Adjusted the pH of the mass to 3 with concentrated hydrochloric acid and stirred for 1 hour to 1.5 hours at 10-20° C. Raised the mass temperature to 20-25° C. and stirred. Filtered the solid, washed with water and dried at 80-85° C. for 12-14 hours.
Moisture content: <0.5%
Yield: 60 gm (98.3% wt/wt)
Chromatographic Purity (by HPLC): 99.92%

### Example 2

### Preparation of Tafamidis Form HN2

Charged Tafamidis Meglumine (100 gm) and DM water (1500 ml) and cooled the mass to 10-20° C. Adjusted the pH of the mass to 3.5 with concentrated hydrochloric acid and stirred for 1 hour to 1.5 hours at 10-20° C. Raised the mass temperature to 20-25° C. and stirred. Filtered the solid, washed with water and dried at below 50° C. for 18-24 hours.
Moisture content: 5%
Yield: 60 gm (98.3% wt/wt)
Chromatographic Purity (by HPLC): 99.96%

### Example 3

### Preparation of Tafamidis Form HN3

Charged Tafamidis Meglumine (100 gm) and DM water (1000 ml) and cooled the mass to 10-20° C. Adjusted the pH of the mass to 3 with concentrated hydrochloric acid (100 ml) and stirred for 2 hours to 3 hours at 10-20° C. Filtered the solid, washed with water and dried at below 40° C. under hot air for 14-15 hours.
Moisture content: 5.8%
Yield: 60 gm (98.3% wt/wt)
Chromatographic Purity (by HPLC): 99.94%

### Example 4

### Preparation of Tafamidis Form HN3

Charged Tafamidis Meglumine (100 gm) and DM water (1000 ml) and cooled the mass to 10-15° C. Adjusted the pH of the mass to 3.5 with sulfuric acid (40 ml) at below 15° C. and stirred for 2 hours to 3 hours at 10-20° C. Filtered the solid, washed with water and dried at below 40° C. under vacuum for 14-15 hours.
Moisture content: 6%
Yield: 60 gm (98.3% wt/wt)
Chromatographic Purity (by HPLC): 99.99%

1. A Tafamidis crystalline Form designated as Form HN1 and having a Powder X-Ray Diffraction pattern as shown in FIG. 1.

2. A process for the preparation of the Tafamidis crystalline Form HN1 according to claim 1 having the Powder X-Ray Diffraction pattern as shown in FIG. 1, which comprises:
a. treating Tafamidis Meglumine with water to provide a mass;
b. adjusting a pH of the mass with an acid to 3-4.5 to precipitate a solid; and
c. filtering the solid and drying the solid at 80-85° C. until a moisture content is <1.0% w/w,
wherein the acid is a member selected from group consisting of hydrochloric acid, sulfuric acid, acetic acid, formic acid, trifluoroacetic acid, methanesulfonic acid, and citric acid.

3. A Tafamidis crystalline Form designated as Form HN2 and having a Powder X-Ray Diffraction pattern as shown in FIG. 2.

4. A process for the preparation of the Tafamidis crystalline Form HN2 according to claim 3 having the Powder X-Ray Diffraction pattern as shown in FIG. 2, which comprises:
a. treating Tafamidis Meglumine with water to provide a mass;
b. adjusting a pH of the mass with an acid to 3-4.5 to precipitate a solid; and
c. filtering the solid and drying the solid below 50° C. until a moisture content is 4-6% w/w,
wherein the acid is a member selected from group consisting of hydrochloric acid, sulfuric acid, acetic acid, formic acid, trifluoroacetic acid, methanesulfonic acid, and citric acid.

5. A Tafamidis crystalline Form designated as Form HN3 and having a Powder X-Ray Diffraction pattern as shown in FIG. 3.

6. A process for the preparation of the Tafamidis crystalline Form HN3 according to claim 5 having the Powder X-Ray Diffraction pattern as shown in FIG. 3, which comprises:
a. treating Tafamidis Meglumine with water to provide a mass;
b. adjusting a pH of the mass with an acid to 3-5 to precipitate a solid; and
c. filtering the solid and drying the solid at below 40° C.,
wherein the acid is a member selected from group consisting of hydrochloric acid, sulfuric acid, acetic acid, formic acid, trifluoroacetic acid, methanesulfonic acid, and citric acid.

7. A pharmaceutical composition comprising an oral dosage form of Tafamidis crystalline Form HN1, Form HN2, or Form HN3, wherein the oral dosage form is a tablet, a pill, or a capsule.

8. The process of claim 2, which has a yield greater than 98% wt/wt and a purity greater than 99% by HPLC.

9. The process of claim 4, which has a yield greater than 98% wt/wt and a purity greater than 99% by HPLC.

10. The process of claim 6, which has a yield greater than 98% wt/wt and a purity greater than 99% by HPLC.
